(19) United States
(12) Patent Application Publication
(10) Pub. No.: US 2021/0070779 A1
(43) Pub. Date: Mar. 11, 2021

(54) BORON-CONTAINING SMALL MOLECULES AS ANTI-INFLAMMATORY AGENTS

(71) Applicant: Anacor Pharmaceuticals, Inc., New York, NY (US)

(72) Inventors: Tsutomu Akama, Sunnyvale, CA (US); Jacob J. Plattner, Berkeley, CA (US)

(21) Appl. No.: 17/066,033

(22) Filed: Oct. 8, 2020

Related U.S. Application Data
(63) Continuation of application No. 16/813,090, filed on Mar. 9, 2020, now abandoned.
Application No. 16/813,090 is a continuation of application No. 16/542,748, filed on Aug. 16, 2019, now abandoned.
Application No. 16/542,748 is a continuation of application No. 16/007,664, filed on Jun. 13, 2018, now abandoned.
Application No. 16/007,664 is a continuation of application No. 14/910,996, filed on Feb. 8, 2016, now abandoned.
(60) Provisional application No. 61/881,343, filed on Sep. 23, 2013.

(51) Int. Cl. C07F 5/02; C07F 5/05

(52) U.S. Cl. CPC C07F 5/025; C07F 5/05

(57) ABSTRACT

Compounds, pharmaceutical formulations, and methods of treating anti-inflammatory conditions are disclosed.

### CROSS-REFERENCE TO RELATED APPLICATIONS

This application is a continuation of U.S. patent application Ser. No. 16/813,090, filed Mar. 9, 2020, which is a continuation of U.S. patent application Ser. No. 16/542,748, filed Aug. 16, 2019, which is a continuation of U.S. patent application Ser. No. 16/007,664, filed Jun. 13, 2018, which is a continuation of U.S. patent application Ser. No. 14/910,996, filed Feb. 8, 2016, which is a 371 U.S. National Phase Application of International Patent Cooperation Treaty Application PCT/US14/56800, filed Sep. 22, 2014, which claims priority from U.S. Provisional Patent Application No. 61/881,343, filed Sep. 23, 2013, the disclosures of which are incorporated herein by reference in its entirety for all purposes.

### BACKGROUND OF THE INVENTION

Boron-containing molecules, such as benzoxaboroles, useful as antiinflammatories, have been described previously, such as in U.S. patent application Ser. Nos. 12/399,015 and 13/015,487. Generally speaking, a benzoxaborole has the following structure and substituent numbering system:

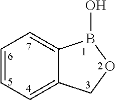

It has now been discovered that particular benzoxaborole classes are surprisingly effective antiinflammatory agents. This, and other uses of these benzoxaboroles are described herein.

Compounds which can inhibit the biological moieties described above, or treat diseases involving those biological moieties, would be a significant advance in the art.

### SUMMARY OF THE INVENTION

This invention provides, among other things, novel compounds useful for treating inflammatory conditions, pharmaceutical compositions containing such compounds, as well as combinations of these compounds with at least one additional therapeutically effective agent.

### DETAILED DESCRIPTION OF THE INVENTION

### I. Definitions and Abbreviations

As used herein, the singular forms “a,” “an”, and “the” include plural references unless the context clearly dictates otherwise. For example, reference to “an active agent” includes a single active agent as well as two or more different active agents in combination. It is to be understood that present teaching is not limited to the specific dosage forms, carriers, or the like, disclosed herein and as such may vary.

The abbreviations used herein generally have their conventional meaning within the chemical and biological arts.

The following abbreviations have been used: Ac is acetyl; AcOH is acetic acid; ACTBr is cetyltrimethylammonium bromide; AIBN is azobisisobutyronitrile or 2,2 azobisisobutyronitrile; aq. is aqueous; Ar is aryl; B₂pin₂ is bis(pinacolato)diboron; Bn is, in general, benzyl [see Cbz for one example of an exception]; (BnS)₂ is benzyl disulfide; BnSH is benzyl thiol or benzyl mercaptan; BnBr is benzyl bromide; Boc is tert-butoxy carbonyl; Boc₂O is di-tert-butyl dicarbonate; Bz is, in general, benzoyl; BzOOH is benzoyl peroxide; Cbz or Z is benzyloxycarbonyl or carboxybenzyl; Cs₂CO₃ is cesium carbonate; CSA is camphor sulfonic acid; CTAB is cetyltrimethylammonium bromide; Cy is cyclohexyl; DABCO is 1,4-diazabicyclo[2.2.2]octane; DCM is dichloromethane or methylene chloride; DHP is dihydropyran; DIAD is diisopropyl azodicarboxylate; DIEA or DIPEA is N,N-diisopropylethylamine; DMAP is 4-(dimethylamino)pyridine; DME is 1,2-dimethoxyethane; DMF is N,N-dimethylformamide; DMSO is dimethylsulfoxide; equiv or eq. is equivalent; EtOAc is ethyl acetate; EtOH is ethanol; Et₂O is diethyl ether; EDCI is N-(3-dimethylaminopropyl)-N′-ethylcarbodiimide hydrochloride; ELS is evaporative light scattering; equiv or eq is equivalent; h is hours; HATU is O-(7-azabenzotriazol-1-yl)-N,N,N′,N′-tetramethyluronium hexafluorophosphate; HOBt is N-hydroxybenzotriazole; HCl is hydrochloric acid; HPLC is high pressure liquid chromatography; ISCO Companion is automated flash chromatography equipment with fraction analysis by UV absorption available from Presearch; KOAc or AcOK is potassium acetate; K₂CO₃ is potassium carbonate; LiAlH₄ or LAH is lithium aluminum hydride; LDA is lithium diisopropylamide; LHMDS is lithium bis(trimethylsilyl) amide; KHMDS is potassium bis(trimethylsilyl) amide; LiOH is lithium hydroxide; m-CPBA is 3-chloroperoxybenzoic acid; MeCN or ACN is methyl cyanide or cyanomethane or ethanenitrile or acetonitrile which are all names for the same compound; MeOH is methanol; MgSO₄ is magnesium sulfate; mins or min is minutes; Mp or MP is melting point; NaCNBH₃ is sodium cyanoborohydride; NaOH is sodium hydroxide; Na₂SO₄ is sodium sulfate; NBS is N-bromosuccinimide; NH₄Cl is ammonium chloride; NIS is N-iodosuccinimide; N₂ is nitrogen; NMM is N-methylmorpholine; n-BuLi is n-butyllithium; overnight is O/N; PdCl₂(pddf) is 1,1′-Bis(diphenylphosphino) ferrocene]dichloropalladium(II); Pd/C is the catalyst known as palladium on carbon; Pd₂(dba)₃ is an organometallic catalyst known as tris(dibenzylideneacetone) dipalladium(0); Ra Ni or Raney Ni is Raney nickel; Ph is phenyl; PMB is p-methoxybenzyl; PrOH is 1-propanol; iPrOH is 2-propanol; POCl₃ is phosphorus chloride oxide; PTSA is para-toluene sulfonic acid; Pyr. or Pyr or Py as used herein means Pyridine; RT or rt or r.t. is room temperature; sat. is saturated; Si-amine or Si—NH₂ is amino-functionalized silica, available from SiliCycle; Si-pyr is pyridyl-functionalized silica, available from SiliCycle; TEA or Et₃N is triethylamine; TFA is trifluoroacetic acid; Tf₂O is trifluoromethanesulfonic anhydride; THF is tetrahydrofuran; TFAA is trifluoroacetic anhydride; THP is tetrahydropyranyl; TMSI is trimethylsilyl iodide; H₂O is water; diNO₂PhSO₂Cl is dinitrophenyl sulfonyl chloride; 3-F-4-NO₂-PhSO₂Cl is 3-fluoro-4-nitrophenylsulfonyl chloride; 2-MeO-4-NO₂-PhSO₂Cl is 2-methoxy-4-nitrophenylsulfonyl chloride; and (EtO)₂POCH₂COOEt is a triethylester of phosphonoacetic acid known as triethyl phosphonoacetate.

“Compound of the invention,” as used herein refers to the compounds discussed herein, salts (e.g. pharmaceutically acceptable salts), prodrugs, solvates and hydrates of these compounds.

“Combination of the invention,” as used herein refers to the compounds and antiprotozoals discussed herein as well as acids, bases, salt forms (such as pharmaceutically acceptable salts), prodrugs, solvates and hydrates of these compounds and antiprotozoals.

“Boron containing compounds”, as used herein, refers to the compounds of the invention that contain boron as part of their chemical formula.

Where substituent groups are specified by their conventional chemical formulae, written from left to right, they equally encompass the chemically identical substituents, which would result from writing the structure from right to left, e.g., —CH₂O— is intended to also recite —OCH₂—.

The term “poly” as used herein means at least 2. For example, a polyvalent metal ion is a metal ion having a valency of at least 2.

“Moiety” refers to a radical of a molecule that is attached to the remainder of the molecule.

The symbol [symbol] , whether utilized as a bond or displayed perpendicular to a bond, indicates the point at which the displayed moiety is attached to the remainder of the molecule.

The term “alkyl,” by itself or as part of another substituent, means, unless otherwise stated, a straight or branched chain, or cyclic hydrocarbon radical, or combination thereof, which may be fully saturated, mono- or polyunsaturated and can include di- and multivalent radicals, having the number of carbon atoms designated (i.e. C₁-C₁₀ means one to ten carbons). In some embodiments, the term “alkyl” means a straight or branched chain, or combinations thereof, which may be fully saturated, mono- or polyunsaturated and can include di- and multivalent radicals. Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers.

The term “alkylene” by itself or as part of another substituent means a divalent radical derived from an alkane, as exemplified, but not limited, by —CH₂CH₂CH₂CH₂—, and further includes those groups described below as “heteroalkylene.” Typically, an alkyl (or alkylene) group will have from 1 to 24 carbon atoms, with those groups having 10 or fewer carbon atoms being preferred in the invention. A “lower alkyl” or “lower alkylene” is a shorter chain alkyl or alkylene group, generally having eight or fewer carbon atoms.

The term “alkenylene” by itself or as part of another substituent means a divalent radical derived from an alkene.

The term “cycloalkylene” by itself or as part of another substituent means a divalent radical derived from a cycloalkane.

The term “heteroalkylene” by itself or as part of another substituent means a divalent radical derived from an heteroalkane.

The term “heterocycloalkylene” by itself or as part of another substituent means a divalent radical derived from an heterocycloalkane.

The term “arylene” by itself or as part of another substituent means a divalent radical derived from an aryl.

The term “heteroarylene” by itself or as part of another substituent means a divalent radical derived from heteroaryl.

The terms “alkoxy,” “alkylamino” and “alkylthio” (or thioalkoxy) are used in their conventional sense, and refer to those alkyl groups attached to the remainder of the molecule via an oxygen atom, an amino group, or a sulfur atom, respectively.

The term “heteroalkyl,” by itself or in combination with another term, means, unless otherwise stated, a stable straight or branched chain, or cyclic hydrocarbon radical, or combinations thereof, consisting of the stated number of carbon atoms and at least one heteroatom. In some embodiments, the term “heteroalkyl,” by itself or in combination with another term, means a stable straight or branched chain, or combinations thereof, consisting of the stated number of carbon atoms and at least one heteroatom. In an exemplary embodiment, the heteroatoms can be selected from the group consisting of B, O, N and S, and wherein the nitrogen and sulfur atoms may optionally be oxidized and the nitrogen heteroatom may optionally be quaternized. The heteroatom(s) B, O, N and S may be placed at any interior position of the heteroalkyl group or at the position at which the alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —CH₂—CH═N—OCH₃, and —CH═CH—N(CH₃)—CH₃. Up to two heteroatoms may be consecutive, such as, for example, —CH₂—NH—OCH₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “halo(C₁-C₄)alkyl” is mean to include, but not be limited to, trifluoromethyl, 2,2,2-trifluoroethyl, 4-chlorobutyl, 3-bromopropyl, and the like.

The term “aryl” means, unless otherwise stated, a polyunsaturated, aromatic, substituent that can be a single ring or multiple rings (preferably from 1 or 2 or 3 rings), which are fused together or linked covalently. The term “heteroaryl” refers to aryl groups (or rings) that contain from one to four heteroatoms. In an exemplary embodiment, the heteroatom is selected from B, N, O, and S, wherein the nitrogen and sulfur atoms are optionally oxidized, and the nitrogen atom(s) are optionally quaternized. A heteroaryl group can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

Each of the above terms (e.g., “alkyl,” “heteroalkyl,” “aryl” and “heteroaryl”) are meant to include both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) are generically referred to as “alkyl group substituents,” and they can be one or more of a variety of groups selected from, but not limited to: —R′, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR′″″—C(NR′R″R′″)═NR″″, —NR″″—C(NR′R″)═NR′″, —S(O)R′, —S(O)₂R′, —S(O)₂NR′R″, —NR″SO₂R′, —CN, —NO₂, —N₃, —CH(Ph)₂, fluoro(C₁-C₄)alkoxy, and fluoro(C₁-C₄)alkyl, in a number ranging from zero to (2m′+1), where m′ is the total number of carbon atoms in such radical. R′, R″, R′″, R″″ and R′″″ each preferably independently refer to hydrogen, substituted or unsubstituted heteroalkyl, substituted or unsubstituted aryl, e.g., aryl substituted with 1-3 halogens, substituted or unsubstituted alkyl, alkoxy or thioalkoxy groups, or arylalkyl groups. When a compound of the invention includes more than one R group, for example, each of the R groups is independently selected as are each R′, R″, R′″, R″″ and R′″″ groups when more than one of these groups is present. When R′ and R″ are attached to the same nitrogen atom, they can be combined with the nitrogen atom to form a 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are generically referred to as “aryl group substituents.” The substituents are selected from, for example: —R′, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR′″″—C(NR′R″R′″)═NR″″, —NR″″—C(NR′R″)═NR′″, —S(O)R′, —S(O)₂R′, —S(O)₂NR′R″, —NR″SO₂R′, —CN, —NO₂, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″, R″″ and R′″″ are preferably independently selected from hydrogen, substituted or unsubstituted alkyl, substituted or unsubstituted heteroalkyl, substituted or unsubstituted aryl and substituted or unsubstituted heteroaryl. When a compound of the invention includes more than one R group, for example, each of the R groups is independently selected as are each R′, R″, R′″, R″″ and R′″″ groups when more than one of these groups is present.

Two of the substituents on adjacent atoms of the aryl or heteroaryl ring may optionally be replaced with a substituent of the formula -T-C(O)—(CRR′)_{q}—U—, wherein T and U are independently —NR—, —O—, —CRR′— or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′— or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X—(CR″R′″)_{d}—, where s and d are independently integers of from 0 to 3, and X is —O—, —NR′—, —S—, —S(O)—, —S(O)₂—, or —S(O)₂NR′—. The substituents R, R′, R″ and R′″ are preferably independently selected from hydrogen or substituted or unsubstituted C₁ or C₂ or C₃ or C₄ or C₅ or C₆ alkyl.

“Ring” as used herein, means a substituted or unsubstituted cycloalkyl, substituted or unsubstituted heterocycloalkyl, substituted or unsubstituted aryl, or substituted or unsubstituted heteroaryl. A ring includes fused ring moieties. The number of atoms in a ring is typically defined by the number of members in the ring. For example, a “5- to 7-membered ring” means there are 5 or 6 or 7 atoms in the encircling arrangement. Unless otherwise specified, the ring optionally includes a heteroatom. Thus, the term “5- to 7-membered ring” includes, for example phenyl, pyridinyl and piperidinyl. The term “5- to 7-membered heterocycloalkyl ring”, on the other hand, would include pyridinyl and piperidinyl, but not phenyl. The term “ring” further includes a ring system comprising more than one “ring”, wherein each “ring” is independently defined as above.

As used herein, the term “heteroatom” includes atoms other than carbon (C) and hydrogen (H). Examples include oxygen (O), nitrogen (N) sulfur (S), silicon (Si), germanium (Ge), aluminum (Al) and boron (B).

The term “leaving group” means a functional group or atom which can be displaced by another functional group or atom in a substitution reaction, such as a nucleophilic substitution reaction. By way of example, representative leaving groups include triflate, chloro, bromo and iodo groups; sulfonic ester groups, such as mesylate, tosylate, brosylate, nosylate and the like; and acyloxy groups, such as acetoxy, trifluoroacetoxy and the like.

The symbol “R” is a general abbreviation that represents a substituent group that is selected from substituted or unsubstituted alkyl, substituted or unsubstituted heteroalkyl, substituted or unsubstituted aryl, substituted or unsubstituted heteroaryl, substituted or unsubstituted cycloalkyl and substituted or unsubstituted heterocycloalkyl groups.

By “effective” amount of a drug, formulation, or permeant is meant a sufficient amount of an active agent to provide the desired local or systemic effect. A “Topically effective,” “pharmaceutically effective,” or “therapeutically effective” amount refers to the amount of drug needed to effect the desired therapeutic result.

“Topically effective” refers to a material that, when applied to the skin, nail, hair, claw or hoof produces a desired pharmacological result either locally at the place of application or systemically as a result of transdermal passage of an active ingredient in the material.

The term “pharmaceutically acceptable salt” is meant to include a salt of a compound of the invention which is prepared with relatively nontoxic acids or bases, depending on the particular substituents found on the compounds described herein. When compounds of the invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino (such as choline or diethylamine or amino acids such as d-arginine, l-arginine, d-lysine, l-lysine), or magnesium salt, or a similar salt. When compounds of the invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science 66: 1-19 (1977)). Certain specific compounds of the invention contain both basic and acidic functionalities that allow the compounds to be converted into either base or acid addition salts.

The neutral forms of the compounds are preferably regenerated by contacting the salt with a base or acid and isolating the parent compounds in the conventional manner. The parent form of the compound differs from the various salt forms in certain physical properties, such as solubility in polar solvents.

In addition to salt forms, the invention provides compounds which are in a prodrug form. Prodrugs of the compounds described herein readily undergo chemical changes under physiological conditions to provide the compounds of the invention. Additionally, prodrugs can be converted to the compounds of the invention by chemical or biochemical methods in an ex vivo environment.

Certain compounds of the invention can exist in unsolvated forms as well as solvated forms, including hydrated forms. In general, the solvated forms are equivalent to unsolvated forms and are encompassed within the scope of the invention. Certain compounds of the invention may exist in multiple crystalline or amorphous forms.

Certain compounds of the invention possess asymmetric carbon atoms (optical centers) or double bonds; the racemates, diastereomers, geometric isomers and individual isomers are encompassed within the scope of the invention. The graphic representations of racemic, ambiscalemic and scalemic or enantiomerically pure compounds used herein are taken from Maehr, J. Chem. Ed. 1985, 62: 114-120. Solid and broken wedges are used to denote the absolute configuration of a stereocenter unless otherwise noted. When the compounds described herein contain olefinic double bonds or other centers of geometric asymmetry, and unless specified otherwise, it is intended that the compounds include both E and Z geometric isomers. Likewise, all tautomeric forms are included.

Compounds of the invention can exist in particular geometric or stereoisomeric forms. The invention contemplates all such compounds, including cis- and trans-isomers, (−)- and (+)-enantiomers, (R)- and (S)-enantiomers, diastereomers, (D)-isomers, (L)-isomers, the racemic mixtures thereof, and other mixtures thereof, such as enantiomerically or diastereomerically enriched mixtures, as falling within the scope of the invention. Additional asymmetric carbon atoms can be present in a substituent such as an alkyl group. All such isomers, as well as mixtures thereof, are intended to be included in this invention.

Optically active (R)- and (S)-isomers and d and l isomers can be prepared using chiral synthons or chiral reagents, or resolved using conventional techniques. If, for instance, a particular enantiomer of a compound of the invention is desired, it can be prepared by asymmetric synthesis, or by derivatization with a chiral auxiliary, where the resulting diastereomeric mixture is separated and the auxiliary group cleaved to provide the pure desired enantiomers. Alternatively, where the molecule contains a basic functional group, such as an amino group, or an acidic functional group, such as a carboxyl group, diastereomeric salts can be formed with an appropriate optically active acid or base, followed by resolution of the diastereomers thus formed by fractional crystallization or chromatographic means known in the art, and subsequent recovery of the pure enantiomers. In addition, separation of enantiomers and diastereomers is frequently accomplished using chromatography employing chiral, stationary phases, optionally in combination with chemical derivatization (e.g., formation of carbamates from amines).

The compounds of the invention may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). The compounds may also be labeled with stable isotopes such as deuterium. All isotopic variations of the compounds of the invention, whether radioactive or not, are intended to be encompassed within the scope of the invention.

The term “pharmaceutically acceptable carrier” or “pharmaceutically acceptable vehicle” refers to any formulation or carrier medium that provides the appropriate delivery of an effective amount of an active agent as defined herein, does not interfere with the effectiveness of the biological activity of the active agent, and that is sufficiently non-toxic to the animal. Representative carriers include water, oils, both vegetable and mineral, cream bases, lotion bases, ointment bases and the like. These bases include suspending agents, thickeners, penetration enhancers, and the like. Their formulation is well known to those in the pharmaceutical arts. Additional information concerning carriers can be found in Remington: The Science and Practice of Pharmacy, 21st Ed., Lippincott, Williams & Wilkins (2005) which is incorporated herein by reference.

The term “pharmaceutically acceptable additive” refers to preservatives, antioxidants, fragrances, emulsifiers, dyes and excipients known or used in the field of drug formulation and that do not unduly interfere with the effectiveness of the biological activity of the active agent, and that is sufficiently non-toxic to the animal. Additives for topical formulations are well-known in the art, and may be added to the topical composition, as long as they are pharmaceutically acceptable and not deleterious to the epithelial cells or their function. Further, they should not cause deterioration in the stability of the composition. For example, inert fillers, anti-irritants, tackifiers, excipients, fragrances, opacifiers, antioxidants, gelling agents, stabilizers, surfactant, emollients, coloring agents, preservatives, buffering agents, other permeation enhancers, and other conventional components of topical or transdermal delivery formulations as are known in the art.

The terms “enhancement,” “penetration enhancement” or “permeation enhancement” relate to an increase in the permeability of the skin, nail, hair, claw or hoof to a drug, so as to increase the rate at which the drug permeates through the skin, nail, hair, claw or hoof. The enhanced permeation effected through the use of such enhancers can be observed, for example, by measuring the rate of diffusion of the drug through animal skin, nail, hair, claw or hoof using a diffusion cell apparatus. A diffusion cell is described by Merritt et al. Diffusion Apparatus for Skin Penetration, J of Controlled Release, 1 (1984) pp. 161-162. The term “permeation enhancer” or “penetration enhancer” intends an agent or a mixture of agents, which, alone or in combination, act to increase the permeability of the skin, nail, hair or hoof to a drug.

The term “excipients” is conventionally known to mean carriers, diluents and/or vehicles used in formulating drug compositions effective for the desired use.

The terms “effective amount” or a “therapeutically effective amount” of a drug or pharmacologically active agent refers to a nontoxic but sufficient amount of the drug or agent to provide the desired effect. In the oral dosage forms of the present disclosure, an “effective amount” of one active of the combination is the amount of that active that is effective to provide the desired effect when used in combination with the other active of the combination. The amount that is “effective” will vary from subject to subject, depending on the age and general condition of the individual, the particular active agent or agents, and the appropriate “effective” amount in any individual case may be determined by one of ordinary skill in the art using routine experimentation.

The phrases “active ingredient”, “therapeutic agent”, “active”, or “active agent” mean a chemical entity which can be effective in treating a targeted disorder, disease or condition.

The phrase “pharmaceutically acceptable” means moieties or compounds that are, within the scope of medical judgment, suitable for use in humans without causing undesirable biological effects such as undue toxicity, irritation, allergic response, and the like, for example.

The phrase “oral dosage form” means any pharmaceutical composition administered to a subject via the oral cavity. Exemplary oral dosage forms include tablets, capsules, films, powders, sachets, granules, solutions, solids, suspensions or as more than one distinct unit (e.g., granules, tablets, and/or capsules containing different actives) packaged together for co-administration, and other formulations known in the art. An oral dosage form can be one, two, three, four, five or six units. When the oral dosage form has multiple units, all of the units are contained within a single package, (e.g. a bottle or other form of packaging such as a blister pack). When the oral dosage form is a single unit, it may or may not be in a single package. In a preferred embodiment, the oral dosage form is one, two or three units. In a particularly preferred embodiment, the oral dosage form is one unit.

The phrase “unit”, as used herein, refers to the number of discrete objects to be administered which comprise the dosage form. In some embodiments, the dosage form includes a compound of the invention in one capsule. This is a single unit. In some embodiments, the dosage form includes a compound of the invention as part of a therapeutically effective dosage of a cream or ointment. This is also a single unit. In some embodiments, the dosage form includes a compound of the invention and another active ingredient contained within one capsule, or as part of a therapeutically effective dosage of a cream or ointment. This is a single unit, whether or not the interior of the capsule includes multiple discrete granules of the active ingredient. In some embodiments, the dosage form includes a compound of the invention in one capsule, and the active ingredient in a second capsule. This is a two unit dosage form, such as two capsules or tablets, and so such units are contained in a single package. Thus the term ‘unit’ refers to the object which is administered to the animal, not to the interior components of the object.

The term, “prodrug”, as defined herein, is a derivative of a parent drug molecule that exerts its pharmacological effect only after chemical and/or enzymatic conversion to its active form in vivo. Prodrugs include those designed to circumvent problems associated with delivery of the parent drug. This may be due to poor physicochemical properties, such as poor chemical stability or low aqueous solubility, and may also be due to poor pharmacokinetic properties, such as poor bioavailability or poor half-life. Thus, certain advantages of prodrugs may include improved chemical stability, absorption, and/or PK properties of the parent carboxylic acids. Prodrugs may also be used to make drugs more amenable to the animal, by minimizing the frequency (e.g., once daily) or route of dosing (e.g., oral), or to improve the taste or odor if given orally, or to minimize pain if given parenterally.

“Biological medium,” as used herein refers to both in vitro and in vivo biological milieus. Exemplary in vitro “biological media” include, but are not limited to, cell culture, tissue culture, homogenates, plasma and blood. In vivo applications are generally performed in mammals, preferably humans.

“Inhibiting” and “blocking,” are used interchangeably herein to refer to the partial or full blockade of an enzyme, such as a beta-lactamase or a leucyl t-RNA synthetase.

Boron is able to form additional covalent or dative bonds with oxygen, sulfur or nitrogen under some circumstances in this invention.

Embodiments of the invention also encompass compounds that are poly- or multi-valent species, including, for example, species such as dimers, trimers, tetramers and higher homologs of the compounds of use in the invention or reactive analogues thereof.

“Salt counterion”, as used herein, refers to positively charged ions that associate with a compound of the invention when the boron is fully negatively or partially negatively charged. Examples of salt counterions include H⁺, H₃O⁺, ammonium, potassium, calcium, magnesium, organic amino (such as choline or diethylamine or amino acids such as d-arginine, l-arginine, d-lysine, l-lysine), and sodium.

The compounds comprising a boron bonded to a carbon and three heteroatoms (such as three oxygens described in this section) can optionally contain a fully negatively charged boron or partially negatively charged boron. Due to the negative charge, a positively charged counterion may associate with this compound, thus forming a salt. Examples of positively charged counterions include H⁺, H₃O⁺, ammonium, potassium, calcium, magnesium, organic amino (such as choline or diethylamine or amino acids such as d-arginine, l-arginine, d-lysine, l-lysine), and sodium. These salts of the compounds are implicitly contained in descriptions of these compounds.

### II. Introduction

The invention provides novel boron compounds. The novel compounds, as well as pharmaceutical compositions containing such compounds or combinations of these compounds with at least one additional therapeutically effective agent, can be used for, among other things, treating inflammatory conditions.

### III. The Compounds

### III.a) Cyclic Boronic Esters

In one aspect, the invention provides a compound of the invention. In an exemplary embodiment, the invention is a compound described herein. In an exemplary embodiment, the invention is a compound according to a formula described herein.

In another aspect, the invention provides a compound, or a salt thereof, which is 4-(1-hydroxy-1,3-dihydrobenzo[c][1,2]oxaborole-5-carbonyl)benzonitrile. In another aspect, the invention provides a compound, or a salt thereof, which is 4-(hydroxy(1-hydroxy-1,3-dihydrobenzo[c][1,2]oxaborol-5-yl)methyl)benzonitrile. In another aspect, the invention provides a compound, or a salt thereof, which is 4-((1-hydroxy-1,3-dihydrobenzo[c][1,2]oxaborol-5-yl)methyl)benzonitrile. In another aspect, the invention provides a compound, or a salt thereof, which is 4-(1-hydroxy-1,3-dihydrobenzo[c][1,2]oxaborol-5-ylsulfinyl)benzonitrile. In another aspect, the invention provides a compound, or a salt thereof, which is 4-(1-hydroxy-1,3-dihydrobenzo[c][1,2]oxaborol-5-ylsulfinyl)benzonitrile.

In an exemplary embodiment, the invention provides a compound described herein, or a salt, hydrate or solvate thereof, or a combination thereof. In an exemplary embodiment, the invention provides a compound described herein, or a salt, hydrate or solvate thereof. In an exemplary embodiment, the invention provides a compound described herein, or a salt thereof. In an exemplary embodiment, the salt is a pharmaceutically acceptable salt. In an exemplary embodiment, the invention provides a compound described herein, or a hydrate thereof. In an exemplary embodiment, the invention provides a compound described herein, or a solvate thereof. In an exemplary embodiment, the invention provides a compound described herein, or a prodrug thereof. In an exemplary embodiment, the invention provides a salt of a compound described herein. In an exemplary embodiment, the invention provides a pharmaceutically acceptable salt of a compound described herein. In an exemplary embodiment, the invention provides a hydrate of a compound described herein. In an exemplary embodiment, the invention provides a solvate of a compound described herein. In an exemplary embodiment, the invention provides a prodrug of a compound described herein.

In an exemplary embodiment, alkyl is linear alkyl. In another exemplary embodiment, alkyl is branched alkyl.

In an exemplary embodiment, heteroalkyl is linear heteroalkyl. In another exemplary embodiment, heteroalkyl is branched heteroalkyl.

### III.b) Combinations Comprising Additional Therapeutic Agents

The compounds of the invention may also be used in combination with additional therapeutic agents. The invention thus provides, in a further aspect, a combination comprising a compound described herein or a pharmaceutically acceptable salt thereof together with at least one additional therapeutic agent. In an exemplary embodiment, the additional therapeutic agent is a compound of the invention. In an exemplary embodiment, the additional therapeutic agent includes a boron atom. In an exemplary embodiment, the additional therapeutic agent does not contain a boron atom.

The compounds of the invention may also be used in combination with additional therapeutic agents. The invention thus provides, in a further aspect, a combination comprising a compound described herein or a pharmaceutically acceptable salt thereof together with at least one additional therapeutic agent. In an exemplary embodiment, the additional therapeutic agent is a compound of the invention. In an exemplary embodiment, the additional therapeutic agent includes a boron atom. In an exemplary embodiment, the additional therapeutic agent does not contain a boron atom. In an exemplary embodiment, the additional therapeutic agent is a compound described in sections III a)-d).

When a compound of the invention is used in combination with a second therapeutic agent active against the same disease state, the dose of each compound may differ from that when the compound is used alone. Appropriate doses will be readily appreciated by those skilled in the art. It will be appreciated that the amount of a compound of the invention required for use in treatment will vary with the nature of the condition being treated and the age and the condition of the patient and will be ultimately at the discretion of the attendant physician or veterinarian. In an exemplary embodiment, the additional therapeutic agent is an antiinflammatory. In an exemplary embodiment, the additional therapeutic agent is a steroid or cyclosporine or psoralen or UVA or retinoid or methotrexate or vitamin D₃ analog. In an exemplary embodiment, the steroid is a systemic steroid or a topical steroid. In an exemplary embodiment, the additional therapeutic agent is topical steroid or antihistamine or calcineurin inhibitor. In an exemplary embodiment, the additional therapeutic agent is a corticosteroid or an NSAID. In an exemplary embodiment, the additional therapeutic agent is a PDE4 inhibitor. In an exemplary embodiment, the additional therapeutic agent is rolipram or roflumilast or apremilast.

In an exemplary embodiment, the additional therapeutic agent is cyclosporine. In an exemplary embodiment, the additional therapeutic agent is an anti-TNF antibody. In an exemplary embodiment, the additional therapeutic agent is selected from the group consisting of infliximab (Remicade), adalimumab (Humira), certolizumab pegol (Cimzia), and golimumab (Simponi). In an exemplary embodiment, the additional therapeutic agent is a circulating receptor fusion protein. In an exemplary embodiment, the additional therapeutic agent is etanercept (Enbrel). In an exemplary embodiment, the additional therapeutic agent is an antibody which specifically targets IL-23. In an exemplary embodiment, the additional therapeutic agent is ustekinumab. In an exemplary embodiment, the additional therapeutic agent is an antibody which specifically targets the LFA-1 binder. In an exemplary embodiment, the additional therapeutic agent is Efalizumab.

The individual components of such combinations may be administered either simultaneously or sequentially in a unit dosage form. The unit dosage form may be a single or multiple unit dosage forms. In an exemplary embodiment, the invention provides a combination in a single unit dosage form. An example of a single unit dosage form is a capsule wherein both the compound of the invention and the additional therapeutic agent are contained within the same capsule. In an exemplary embodiment, the invention provides a combination in a two unit dosage form. An example of a two unit dosage form is a first capsule which contains the compound of the invention and a second capsule which contains the additional therapeutic agent. Thus the term ‘single unit’ or ‘two unit’ or ‘multiple unit’ refers to the object which the animal (such as a human) ingests, not to the interior components of the object. Appropriate doses of known therapeutic agents will be readily appreciated by those skilled in the art.

The combinations referred to herein may conveniently be presented for use in the form of a pharmaceutical formulation. Thus, an exemplary embodiment of the invention is a pharmaceutical formulation comprising a) a compound of the invention; b) an additional therapeutic agent and c) a pharmaceutically acceptable excipient. In an exemplary embodiment, the pharmaceutical formulation is a unit dosage form. In an exemplary embodiment, the pharmaceutical formulation is a single unit dosage form. In an exemplary embodiment, the pharmaceutical formulation is a two unit dosage form. In an exemplary embodiment, the pharmaceutical formulation is a two unit dosage form comprising a first unit dosage form and a second unit dosage form, wherein the first unit dosage form includes a) a compound of the invention and b) a first pharmaceutically acceptable excipient; and the second unit dosage form includes c) an additional therapeutic agent and d) a second pharmaceutically acceptable excipient.

It is to be understood that the invention covers all combinations of aspects and/or embodiments, as well as suitable, convenient and preferred groups described herein.

### III.c) Preparation of Boron-Containing Compounds

Compounds of use in the invention can be prepared using commercially available starting materials, known intermediates, or by using the synthetic methods described herein, or published in references described and incorporated by reference herein, such as U.S. Prov. Pat. App. 60/654,060; Filed Feb. 16, 2005; U.S. patent application Ser. No. 11/357,687, Filed Feb. 16, 2006; U.S. patent application Ser. No. 11/505,591, Filed Aug. 16, 2006, U.S. Prov. Pat. App. 60/823,888 filed on Aug. 29, 2006 and 60/774,532 filed on Feb. 16, 2006; U.S. patent application Ser. No. 11/676,120, Filed Feb. 16, 2007; U.S. patent application Ser. No. 11/676,120, Filed Feb. 16, 2007; U.S. patent application Ser. No. 12/399,015, filed Mar. 5, 2009; U.S. patent application Ser. No. 13/015,487, filed Jan. 27, 2011, which are herein incorporated by reference in their entirety for all purposes. Methods of producing the compounds of the invention are also described in these patent applications.

Compounds described herein can be converted into hydrates and solvates by methods similar to those described herein.

### IV. The Methods

In another aspect of the invention, the compounds of the invention can be utilized in the methods described herein. In an exemplary embodiment, in any of the methods described herein, the organism being administered the compound of the invention is not otherwise in need of being administered said compound of the invention. In an exemplary embodiment, in any of the methods described herein, the animal being administered the compound of the invention is not otherwise in need of treatment with said compound of the invention.

a) Decreasing the Production of a Cytokine and/or Chemokine

In another aspect, the invention provides a method for decreasing the production of a cytokine and/or a chemokine, the method comprising: contacting a cell with a compound of the invention, wherein production of the cytokine and/or chemokine by the cell is decreased. In another aspect, the invention provides a method for decreasing the production of a cytokine and/or a chemokine, the method comprising: contacting a cell with a compound described herein or a pharmaceutically acceptable salt thereof, wherein production of the cytokine and/or chemokine by the cell is decreased. In an exemplary embodiment, the compound of the invention is a compound described herein, or a pharmaceutically acceptable salt thereof. In an exemplary embodiment, the compound of the invention is a compound described herein. In an exemplary embodiment, the cell is contacted with a therapeutically effective amount of the compound. In an exemplary embodiment, the compound is according to a formula described herein.

In an exemplary embodiment, the method is for decreasing the production of a cytokine, which is a TH1 cytokine. In an exemplary embodiment, the TH1 cytokine is IFN-γ or IL-2.

In an exemplary embodiment, the method is for decreasing the production of a cytokine, which is a TH2 cytokine. In an exemplary embodiment, the TH2 cytokine is selected from the group consisting of IL-4, IL-5, and IL-10.

In an exemplary embodiment, the method is for decreasing the production of a cytokine, which is selected from the group consisting of IL-α, IL-1β, IL-2, IL-3, IL-6, IL-7, IL-9, IL-12, IL-17, IL-18, IL-23, TNF-α, LT, LIF, Oncostatin, IFNα, IFNβ and IFN-γ. In another exemplary embodiment, the cytokine is selected from the group consisting of IL-2, IL-3, IL-6, IL-7, IL-9, IL-12, IL-23, TNF-α, LT, LIF, Oncostatin, and IFN-γ. In another exemplary embodiment, the cytokine is selected from the group consisting of IL-2, IL-23, TNF-α and IFN-γ. In another exemplary embodiment, the cytokine is TNF-α.

In an exemplary embodiment, the method is for decreasing the production of a cytokine, which is selected from the group consisting of IL-2, IL-4, IL-5, IL-6, IL-8, IL-10, IL-12, IL-23, TNF-α and IFN-γ. In an exemplary embodiment, the method is for decreasing the production of IL-4. In an exemplary embodiment, the method is for decreasing the production of IL-23.

In an exemplary embodiment, the method is for decreasing the production of a cytokine, which is selected from the group consisting of IL-4, IL-10, IL-11, W-13 and TGF-β.

In an exemplary embodiment, the method is for decreasing the production of a chemokine, which is selected from the group consisting of IL-8, Gro-α, MIP-1, MCP-1, PGE2, ENA-78, and RANTES. In an exemplary embodiment, the chemokine is MCP-1 or PGE2.

In an exemplary embodiment, for any of the methods described herein, the compound of the invention is present in an amount which will inhibit the production of a cytokine and/or a chemokine by at least about 5 to about 100%, or at least about 30 to about 100%, 40 to about 100%, or at least about 50 to about 100%, or at least about 60 to about 100%, or at least about 70 to about 100%, or at least about 80 to about 100%, or at least about 90 to about 100%, or at least about 30 to about 70%, or at least about 40 to about 90%, or at least about 45 to about 80%, or at least about 55 to about 75%, or at least about 75 to about 98%, or at least about 55 to about 99%, or at least about 5% to about 20% or at least about 10% to about 25%. In an exemplary embodiment, the compound of the invention is a compound described herein.

b) Increasing the Production of a Cytokine and/or a Chemokine

In another aspect, the invention provides a method for increasing the production of a cytokine and/or a chemokine, the method comprising: contacting a cell with a compound of the invention, wherein production of the cytokine and/or chemokine by the cell is increased. In an exemplary embodiment, the compound is described herein or a pharmaceutically acceptable salt thereof. In an exemplary embodiment, the compound of the invention is a compound described herein. In an exemplary embodiment, the cell is contacted with a therapeutically effective amount of the compound. In an exemplary embodiment, the compound is according to a formula described herein.

In an exemplary embodiment, the method is for increasing the production of a cytokine, which is a TH1 cytokine. In an exemplary embodiment, the TH1 cytokine is IFN-γ or IL-2.

In an exemplary embodiment, the method is for increasing the production of a cytokine, which is a TH2 cytokine. In an exemplary embodiment, the TH2 cytokine is selected from the group consisting of IL-4, IL-5 and IL-10.

In an exemplary embodiment, the method is for increasing the production of a cytokine, which is selected from the group consisting of IL-4, IL-10, IL-11, W-13 and TGF-β.

In an exemplary embodiment, the method is for increasing the production of a chemokine, which is selected from the group consisting of IL-8, Gro-α, MIP-1, MCP-1, PGE2, ENA-78, and RANTES. In an exemplary embodiment, the chemokine is MCP-1 or PGE2.

In an exemplary embodiment, for any of the methods described herein, the compound of the invention is present in an amount which will increase the production of a cytokine and/or a chemokine by at least about 5 to about 100%, or at least about 30 to about 100%, 40 to about 100%, or at least about 50 to about 100%, or at least about 60 to about 100%, or at least about 70 to about 100%, or at least about 80 to about 100%, or at least about 90 to about 100%, or at least about 30 to about 70%, or at least about 40 to about 90%, or at least about 45 to about 80%, or at least about 55 to about 75%, or at least about 75 to about 98%, or at least about 55 to about 99%, or at least about 5% to about 20% or at least about 10% to about 25%. In an exemplary embodiment, the compound of the invention is a compound described herein.

c) Decreasing the Release of a Cytokine and/or Chemokine

In another aspect, the invention provides a method for decreasing the release of a cytokine and/or a chemokine, the method comprising: contacting a cell with a compound of the invention, wherein the release of the cytokine and/or chemokine by the cell is decreased. In an exemplary embodiment, the compound of the invention is a compound described herein or a pharmaceutically acceptable salt thereof. The compound of the invention is a compound described herein. In an exemplary embodiment, the cell is contacted with a therapeutically effective amount of the compound. In an exemplary embodiment, the compound is according to a formula described herein.

In an exemplary embodiment, the method is for decreasing the release of a cytokine, which is a TH1 cytokine. In an exemplary embodiment, the TH1 cytokine is a IFN-γ or IL-2.

In an exemplary embodiment, the method is for decreasing the release of a cytokine, which is a TH2 cytokine. In an exemplary embodiment, the TH2 cytokine is selected from the group consisting of IL-4, IL-5 and IL-10.

In an exemplary embodiment, the method is for decreasing the release of a cytokine, which is selected from the group consisting of IL-1α, IL-1β, IL-2, IL-3, IL-6, IL-7, IL-9, IL-12, IL-17, IL-18, IL-23, TNF-α, LT, LIF, Oncostatin, IFNα, IFNβ and IFN-γ. In another exemplary embodiment, the cytokine is selected from the group consisting of IL-2, IL-3, IL-6, IL-7, IL-9, IL-12, IL-23, TNF-α, LT, LIF, Oncostatin, and IFN-γ. In another exemplary embodiment, the cytokine is selected from the group consisting of IL-2, IL-23, TNF-α and IFN-γ. In another exemplary embodiment, the cytokine is TNF-α. In another exemplary embodiment, the cytokine is IFN-γ.

In an exemplary embodiment, the method is for decreasing the release of a cytokine, which is selected from the group consisting of IL-1β, IL-2, IL-4, IL-5, IL-6, IL-8, IL-10, IL-12, IL-23, TNF-α and IFN-γ.

In an exemplary embodiment, the compound of the invention decreases the release of IL-1β, IL-2, IL-4, IL-5, IL-6, IL-8, IL-10, IL-12, IL-23, TNF-α and IFN-γ.

In an exemplary embodiment, the method is for decreasing the release of a cytokine, which is selected from the group consisting of IL-4, IL-10, IL-11, W-13 and TGF-β. In an exemplary embodiment, the method is for decreasing the release of IL-4. In an exemplary embodiment, the method is for decreasing the release of IL-23.

In an exemplary embodiment, the method is for decreasing the release of a chemokine, which is selected from the group consisting of IL-8, Gro-α, MIP-1, MCP-1, PGE2, ENA-78, and RANTES. In an exemplary embodiment, the chemokine is MCP-1 or PGE2.

In an exemplary embodiment, the compound of the invention decreases the release of a member selected from the group consisting of TNF-α, IL-2, IFNγ, IL-5, and IL-10. In an exemplary embodiment, the compound of the invention does not substantially decrease the release of IL-1β, IL-6 and IL-8. In an exemplary embodiment, the compound of the invention does not substantially decrease the release of IL-1β. In an exemplary embodiment, the compound of the invention does not substantially decrease the release of IL-4. In an exemplary embodiment, the compound decreases the release of IL-12 and IL-23.

In an exemplary embodiment, for any of the methods described herein, the compound of the invention is present in an amount which will decrease the release of a cytokine and/or a chemokine by at least about 5 to about 100%, or at least about 30 to about 100%, 40 to about 100%, or at least about 50 to about 100%, or at least about 60 to about 100%, or at least about 70 to about 100%, or at least about 80 to about 100%, or at least about 90 to about 100%, or at least about 30 to about 70%, or at least about 40 to about 90%, or at least about 45 to about 80%, or at least about 55 to about 75%, or at least about 75 to about 98%, or at least about 55 to about 99%, or at least about 5% to about 20% or at least about 10% to about 25%. In another exemplary embodiment, the compound of the invention is a compound described herein or a pharmaceutically acceptable salt thereof.

d) Increasing the Release of a Cytokine and/or a Chemokine

In another aspect, the invention provides a method for increasing the production of a cytokine and/or a chemokine, the method comprising: contacting a cell with a compound of the invention, wherein release of the cytokine and/or chemokine by the cell is increased. In an exemplary embodiment, the compound of the invention is a compound described herein or a pharmaceutically acceptable salt thereof. In an exemplary embodiment, the compound is described herein. In an exemplary embodiment, the cell is contacted with a therapeutically effective amount of the compound. In an exemplary embodiment, the compound is according to a formula described herein.

In an exemplary embodiment, the method is for increasing the release of a cytokine, which is a TH1 cytokine. In an exemplary embodiment, the TH1 cytokine is IFN-γ or IL-2.

In an exemplary embodiment, the method is for increasing the release of a cytokine, which is a TH2 cytokine. In an exemplary embodiment, the TH2 cytokine is selected from the group consisting of IL-4, IL-5 and IL-10.

In an exemplary embodiment, the method is for increasing the release of a cytokine, which is selected from the group consisting of IL-4, IL-10, IL-11, W-13 and TGF-β.

In an exemplary embodiment, the method is for increasing the release of a chemokine, which is selected from the group consisting of IL-8, Gro-α, MIP-1, MCP-1, PGE2, ENA-78, and RANTES. In an exemplary embodiment, the chemokine is MCP-1 or PGE2.

In an exemplary embodiment, for any of the methods described herein, the compound of the invention is present in an amount which will increase release of a cytokine and/or a chemokine by at least about 5 to about 100%, or at least about 30 to about 100%, 40 to about 100%, or at least about 50 to about 100%, or at least about 60 to about 100%, or at least about 70 to about 100%, or at least about 80 to about 100%, or at least about 90 to about 100%, or at least about 30 to about 70%, or at least about 40 to about 90%, or at least about 45 to about 80%, or at least about 55 to about 75%, or at least about 75 to about 98%, or at least about 55 to about 99%, or at least about 5% to about 20% or at least about 10% to about 25%. In an exemplary embodiment, the compound of the invention is a compound described herein or a pharmaceutically acceptable salt thereof.

### e) Inhibiting a Phosphodiesterase

In another aspect, the invention provides a method for inhibiting a phosphodiesterase (PDE), the method comprising: contacting the phosphodiesterase with a compound of the invention, wherein the phosphodiesterase is inhibited. In an exemplary embodiment, the compound of the invention is a compound described herein or a pharmaceutically acceptable salt thereof. In an exemplary embodiment, the compound of the invention is a compound described herein. In an exemplary embodiment, the amount of the compound is a therapeutically effective amount. In an exemplary embodiment, the compound is according to a formula described herein.

In an exemplary embodiment, the phosphodiesterase is selected from the group consisting of PDE1, PDE2, PDE3, PDE4, PDE5, PDE6, PDE7, PDE8, PDE9, PDE10 and PDE11. In an exemplary embodiment, the phosphodiesterase is PDE4. In an exemplary embodiment, the PDE4 is selected from the group consisting of PDE4A, PDE4B, PDE4C and PDE4D. In an exemplary embodiment, the PDE4 is PDE4B. In an exemplary embodiment, the phosphodiesterase is PDE7.

In an exemplary embodiment, the invention provides a method for inhibiting a phosphodiesterase4 (PDE4), but not significantly inhibiting at least one PDE which is selected from the group consisting of PDE1, PDE2, PDE3, PDE5 and PDE6, involving contacting a cell with a compound of the invention, thereby providing said inhibition.

In an exemplary embodiment, for any of the methods described herein, the invention, or a compound described by a formula presented herein, is present in an amount which will inhibit a phosphodiesterase described herein by at least about 5 to about 100%, or at least about 30 to about 100%, 40 to about 100%, or at least about 50 to about 100%, or at least about 60 to about 100%, or at least about 70 to about 100%, or at least about 80 to about 100%, or at least about 90 to about 100%, or at least about 30 to about 70%, or at least about 40 to about 90%, or at least about 45 to about 80%, or at least about 55 to about 75%, or at least about 75 to about 98%, or at least about 55 to about 99%, or at least about 5% to about 20% or at least about 10% to about 25%. In an exemplary embodiment, the compound of the invention is a compound described herein or a pharmaceutically acceptable salt thereof.

### f) Conditions and Effects

In another aspect, the invention provides a method of treating and/or preventing a condition, and/or enhancing an effect, in an animal, the method comprising administering to the animal an effective amount of a compound of the invention, thereby treating and/or preventing the condition. In an exemplary embodiment, the compound of the invention is a compound described herein. In an exemplary embodiment, the compound of the invention is a pharmaceutically acceptable salt of a compound described herein. In an exemplary embodiment, the effective amount is an amount effective to treat the condition. In an exemplary embodiment, the effective amount is an amount effective to prevent the condition. In an exemplary embodiment, the animal is not otherwise is need of treatment with the compound of the invention. In an exemplary embodiment, the compound is according to a formula described herein. In another aspect, the invention provides a method of treating a condition in an animal in need of the treatment, the method comprising administering to the animal an amount of a compound of the invention, thereby treating the condition. In another aspect, the invention provides a method of treating a condition in an animal in need of the treatment, the method comprising administering to the animal a therapeutically effective amount of a compound of the invention, thereby treating the condition. In another aspect, the invention provides a method of preventing a condition, in an animal, the method comprising administering to the animal an amount of a compound of the invention, thereby preventing the condition. In another aspect, the invention provides a method of enhancing an effect, in an animal, the method comprising administering to the animal an effective amount of a compound of the invention, thereby enhancing the effect. In an exemplary embodiment, the compound is according to a formula described in the section entitled “Inhibiting a phosphodiesterase”.

In an exemplary embodiment, the condition is a disease. In an exemplary embodiment, the condition is an inflammatory-related condition. In an exemplary embodiment, the condition involves the increase of production of a cytokine and/or a chemokine. In an exemplary embodiment, the condition involves the decrease of production of a cytokine and/or a chemokine. In an exemplary embodiment, the condition involves the increase of release of a cytokine and/or a chemokine. In an exemplary embodiment, the condition involves the decrease of release of a cytokine and/or a chemokine. In an exemplary embodiment, the condition involves the inhibition of a phosphodiesterase. In an exemplary embodiment, the compound is in an amount sufficient to treat the inflammatory-related disease by inhibiting pro-inflammatory cytokine expression or by stimulating anti-inflammatory cytokine expression, but the amount is less than sufficient to substantially inhibit cyclin dependent kinases. In an exemplary embodiment, the condition is mediated by a cytokine. In an exemplary embodiment, the condition is mediated by a chemokine. In an exemplary embodiment, the condition is mediated by a neutrophil. In an exemplary embodiment, the condition is mediated by a phosphodiesterase. In an exemplary embodiment, the condition is mediated by a phosphodiesterase-4. In an exemplary embodiment, the condition is mediated by a phosphodiesterase-7.

In an exemplary embodiment, the condition is a member selected from periodontitis, keratoconjunctivitis sicca, rheumatoid arthritis, osteoarthritis, Crohn's disease, ulcerative colitis, psoriatic arthritis, traumatic arthritis, rubella arthritis, inflammatory bowel disease, multiple sclerosis, psoriasis, graft versus host disease, systemic lupus erythematosus, cutaneous lupus erythematosus, toxic shock syndrome, irritable bowel syndrome, muscle degeneration, allograft rejections, pancreatitis, insulitis, glomerulonephritis, diabetic nephropathy, renal fibrosis, chronic renal failure, gout, leprosy, acute synovitis, Reiter's syndrome, gouty arthritis, Behcet's disease, spondylitis, endometriosis, non-articular inflammatory conditions, such as intervertebral disk syndrome conditions, bursitis, tendonitis, tenosynovitis or fibromyalgic syndrome; and acute or chronic pain, including but not limited to neurological pain, neuropathies, polyneuropathies, diabetes-related polyneuropathies, trauma, migraine, tension and cluster headache, Horton's disease, varicose ulcers, neuralgias, musculo-skeletal pain, osteo-traumatic pain, fractures, algodystrophy, spondyloarthritis, fibromyalgia, phantom limb pain, back pain, vertebral pain, post-surgery pain, herniated intervertebral disc-induced sciatica, cancer-related pain, vascular pain, visceral pain, childbirth, or HIV-related pain. Other cytokine mediated diseases are allergy, a metabolic disease, a chemotherapy/radiation related complication; diabetes type I; diabetes type II; a liver disease; a gastrointestinal disorder; an ophthalmological disease; allergic conjunctivitis; diabetic retinopathy; Sjogren's syndrome; uveitis; a pulmonary disorder, a renal disease; dermatitis; HIV-related cachexia; cerebral malaria; ankylosing spondylitis; leprosy; anemia; fibromyalgia, kidney failure, stroke, chronic heart failure, endotoxemia, reperfusion injury, ischemia reperfusion, myocardial ischemia, restenosis, thrombosis, angiogenesis, Coronary Heart Disease, Coronary Artery Disease, acute coronary syndrome, Takayasu arteritis, cardiac failure such as heart failure, aortic valve stenosis, cardiomyopathy, myocarditis, vasculitis, vascular restenosis, valvular disease or coronary artery bypass; hypercholesterolemia, diseases or conditions related to blood coagulation or fibrinolysis, such as for example, acute venous thrombosis, pulmonary embolism, thrombosis during pregnancy, hemorrhagic skin necrosis, acute or chronic disseminated intravascular coagulation (DIC), clot formation from surgery, long bed rest or long periods of immobilization, venous thrombosis, fulminant meningococcemia, acute thrombotic strokes, acute coronary occlusion, acute peripheral arterial occlusion, massive pulmonary embolism, axillary vein thrombosis, massive iliofemoral vein thrombosis, occluded arterial or venous cannulae, cardiomyopathy, venoocclusive disease of the liver, hypotension, decreased cardiac output, decreased vascular resistance, pulmonary hypertension, diminished lung compliance, leukopenia or thrombocytopenia; or atherosclerosis.

In an exemplary embodiment, the condition is selected from the group consisting of allergic conjunctivitis, uveitis, glaucoma, optic neuritis, retinal ischemia, diabetic retinopathy, laser induced optic damage, or surgery or trauma-induced proliferative vitreoretinopathy.

In an exemplary embodiment, the condition is selected from the group consisting of allergic rhinitis, asthma, adult respiratory distress syndrome, chronic pulmonary inflammation, chronic obstructive pulmonary disease, emphysema, bronchitis, mucus hypersecretion, silicosis, SARS infection and respiratory tract inflammation.

In an exemplary embodiment, the condition is selected from the group consisting of psoriasis, eczema, atopic dermatitis, contact dermatitis, inflammatory alopecia and acne.

In an exemplary embodiment, the condition is a member selected from Guillain-Barre syndrome, Parkinson's disease, Huntington's disease, Alzheimer's disease, amyotrophic lateral sclerosis, multiple sclerosis and other demyelinating diseases, viral and bacterial meningitis, CNS trauma, spinal cord injury, seizures, convulsions, olivopontocerebellar atrophy, AIDS dementia complex, MERRF and MELAS syndromes, Leber's disease, Wemicke's encephalopathy, Rett syndrome, homocystinuria, hyperprolinemia, hyperhomocysteinemia, nonketotic hyperglycinemia, hydroxybutyric aminoaciduria, sulfite oxidase deficiency, combined systems disease, lead encephalopathy, Tourette's syndrome, hepatic encephalopathy, drug addiction, drug tolerance, drug dependency, depression, bipolar depression, attention deficit disorder (ADD), anxiety and schizophrenia, aneurism, or epilepsy.

In an exemplary embodiment, the condition is selected from the group consisting of bone resorption diseases, osteopetrosis, osteoporosis, and osteoarthritis.

In an exemplary embodiment, the condition is selected from the group consisting of diabetes, systemic cachexia, cachexia secondary to infection or malignancy, cachexia secondary to acquired immune deficiency syndrome (AIDS), obesity, anorexia and bulimia nervosa. In an exemplary embodiment, the condition is selected from the group consisting of sepsis, HIV, HCV, malaria, infectious arthritis, leishmaniasis, Lyme disease, cancer, including but not limited to breast cancer, colon cancer, lung cancer, prostate cancer, multiple myeloma, acute myelogenous leukemia, myelodysplastic syndrome, non-Hodgkins lymphoma, follicular lymphoma, Castleman's disease, and drug resistance.

In an exemplary embodiment, the condition is selected from the group consisting of is bronchial asthma, rhinitis, influenza, stroke, myocardial infarction, thermal injury, adult respiratory distress syndrome (ARDS), multiple organ injury secondary to trauma, acute glomerulonephritis, dermatoses with acute inflammatory components, acute purulent meningitis, hemodialysis, leukapheresis, granulocyte transfusion associated syndromes, and necrotizing enterocolitis.

In an exemplary embodiment, the condition is selected from the group consisting of inflammatory bowel disease (IBD), psoriasis, rheumatoid arthritis (RA), multiple sclerosis (MS), neurodegenerative disorder, cardiovascular disease (CVD) and atherosclerosis, and metabolic disease (the metabolic syndrome and diabetes) as well as infection-related inflammation. In an exemplary embodiment, the condition is a neurodegenerative disorder which is selected from the group consisting of Alzheimer's disease and Parkinson disease. In an exemplary embodiment, the condition is inflammatory bowel disease which is Crohn's disease or ulcerative colitis. In an exemplary embodiment, the condition is a gastrointestinal complication. In an exemplary embodiment, the condition is diarrhea. In an exemplary embodiment, the condition is celiac disease or non-specific colitis. In an exemplary embodiment, the condition is a liver disease. In an exemplary embodiment, the condition is selected from the group consisting of an autoimmune hepatitis, hepatitis C, primary biliary cirrhosis, primary sclerosing cholangitis, and fulminant liver failure. In an exemplary embodiment, the condition is a bone disease. In an exemplary embodiment, the condition is osteoporosis. In an exemplary embodiment, the condition is a pulmonary disorder. In an exemplary embodiment, the condition is selected from the group consisting of: allergic rhinitis, asthma, chronic obstructive pulmonary disease, chronic granulomatous inflammation, cystic fibrosis, and sarcoidosis. In an exemplary embodiment, condition is cardiovascular disease. In an exemplary embodiment, the cardiovascular disease is selected from the group consisting of atheroscleotic cardiac disease, congestive heart failure and restenosis. In an exemplary embodiment, the condition is a renal disease. In an exemplary embodiment, the condition is glomerulonephritis or vasculitis. In an exemplary embodiment, the condition is a member selected from post-radiotherapy related disease or atherosclerosis. In yet another embodiment the condition is atopic dermatitis. In yet another embodiment the condition is actinic keratosis.

In an exemplary embodiment, the condition is selected from the group consisting of psoriasis, inflammatory arthritis, rheumatoid arthritis, asthma, chronic bronchitis, inflammatory bowel disease (IBD), chronic obstructive pulmonary disease (COPD), atopic dermatitis, urticaria, allergic rhinitis, allergic conjunctivitis, vernal conjunctivitis, colitis, esoniophilic granuloma, septic shock, reperfusion injury of the myocardium, reperfusion injury of the brain, chronic glomerulonephritis, endotoxic shock, adult respiratory distress syndrome, cystic fibrosis, arterial restenosis, atherosclerosis, keratosis, rheumatoid spondylitis, osteoarthritis, pyresis, diabetes mellitus, pneumoconiosis, chronic obstructive airways disease, toxic contact eczema, allergic contact eczema, atopic eczema, seborrheic eczema, lichen simplex, sunburn, pruritus in the anogenital area, alopecia areata, hypertrophic scars, discoid lupus erythematosus, systemic lupus erythematosus, follicular pyodermas, wide-area pyodermas, endogenous acne, exogenous acne, acne rosacea, Behcet's disease, anaphylactoid purpura nephritis, leukemia, multiple sclerosis, gastrointestinal disease and autoimmune disease. In an exemplary embodiment, the colitis is selected from the group consisting of ulcerative colitis, Crohn's colitis, diversion colitis, ischemic colitis, infectious colitis, fulminant colitis, chemical colitis, microscopic colitis, lymphocytic colitis, and atypical colitis. In an exemplary embodiment, the colitis is ulcerative colitis or Crohn's colitis. In an exemplary embodiment, the condition is sunburn. In an exemplary embodiment, the condition is inflammation caused by sunburn.

In an exemplary embodiment, the condition is psoriasis. In an exemplary embodiment, the condition is plaque psoriasis or flexural psoriasis (inverse psoriasis) or guttate psoriasis or pustular psoriasis or nail psoriasis or psoriatic arthritis or erythrodermic psoriasis. In an exemplary embodiment, the condition is plaque psoriasis. In an exemplary embodiment, the condition is nail psoriasis.

In an exemplary embodiment, the disorder is selected from the group consisting of cognition impairment or decline or memory impairment. In an exemplary embodiment, the memory impairment is due to dementia. In an exemplary embodiment, the patient is suffering from memory impairment due to Alzheimer's disease, schizophrenia, Parkinson's disease, Huntington's disease, Pick's disease, Creutzfeld-Jakob disease, depression, aging, head trauma, stroke, CNS hypoxia, cerebral senility, multiinfaret dementia, an acute neuronal disease, age-related cognitive decline, HIV or a cardiovascular disease.

In an exemplary embodiment, the disorder is spondyloarthropathy. In an exemplary embodiment, the disorder is selected from the group consisting of psoriatic arthritis, reactive arthritis, uveitis, arthritis associated with ulcerative colitis, arthritis associated with Crohn's disease, juvenile SpA (spondylarthropathy), and ankylosing spondylitis.

Compounds such as those described in this invention may also be used to treat various neurological diseases including: to regulate sleep—insomnia; to aid the recovery CNS tissue from ischemia—recovery from stroke, spinal cord injury and aneurysm; to treat depression; to treat psychosis; to treat memory and learning impairment; to treat inflammatory brain diseases—multiple sclerosis or myasthenia gravis; to suppress brain tumor growth.

In an exemplary embodiment, the PDE4 inhibition is enhancing an effect, wherein the enhanced effect is cognition or memory.

In an exemplary embodiment, the invention provides a method for stimulating ovarian follicular growth in a female, comprising administering to a female a medicament comprising a compound of the invention, whereby ovarian follicular growth is stimulated in the female. In an exemplary embodiment, the compound of the invention is a compound described herein or a pharmaceutically acceptable salt thereof. In an exemplary embodiment, the female is undergoing ovulation induction. In an exemplary embodiment, the female is undergoing controlled ovarian hyperstimulation. In an exemplary embodiment, the medicament is administered simultaneously, separately or sequentially with follicle stimulating hormone (FSH), or an agent having FSH activity, or an agent that stimulates endogenous FSH release.

The invention also provides a method of treating an inflammatory-related disease associated with cytokine expression levels, which comprises administering to an animal in need of such treatment the compound of the invention. In an exemplary embodiment, the compound is according to a formula described herein. In an exemplary embodiment, the compound of the invention is a compound described herein or a pharmaceutically acceptable salt thereof.

In an exemplary embodiment, the invention provides a method of treating or preventing an inflammatory-related disease in an animal, the method comprising administering to the animal a therapeutically effective amount of a compound of the invention, wherein the compound is in an amount sufficient to treat the inflammatory-related disease by inhibiting pro-inflammatory cytokine expression or by stimulating anti-inflammatory cytokine expression, but the amount is less than sufficient to substantially inhibit cyclin dependent kinases. In an exemplary embodiment, the compound of the invention is a compound described herein or a pharmaceutically acceptable salt thereof.

In an exemplary embodiment, the invention provides a method for inhibiting the production of an inflammatory cytokine by cells capable of producing the inflammatory cytokine, the method comprises contacting a cell with a therapeutic amount of compound of the invention, wherein production of the inflammatory cytokine by the cells is inhibited. In an exemplary embodiment, the therapeutic amount is sufficient to inhibit the production of the inflammatory cytokine protein between about 50% and about 99%.

In an exemplary embodiment, the invention provides a method for inhibiting an inflammatory response in an animal, the method comprising: contacting the animal with a therapeutic amount of a compound of the invention, wherein the inflammatory response is inhibited.

In an exemplary embodiment, for any of the methods described herein, the animal is selected from the group consisting of human, cattle, deer, reindeer, goat, honey bee, pig, sheep, horse, cow, bull, dog, guinea pig, gerbil, rabbit, cat, camel, yak, elephant, ostrich, otter, chicken, duck, goose, guinea fowl, pigeon, swan, and turkey. In another exemplary embodiment, for any of the methods described herein, the animal is selected from the group consisting of a human, cattle, goat, pig, sheep, horse, cow, bull, dog, guinea pig, gerbil, rabbit, cat, chicken and turkey. In another exemplary embodiment, for any of the methods described herein, the animal is a human.

In an exemplary embodiment, for any of the methods described herein, a compound of the invention and/or a pharmaceutical formulation described herein can be used.

In another exemplary embodiment, in any of the methods described herein, the animal being administered the compound of the invention is not otherwise in need of treatment with the compound of the invention. In another exemplary embodiment, in any of the methods of treating/preventing a condition or enhancing an effect described herein, the animal being administered the compound of the invention is not otherwise in need of treatment with the compound of the invention.

In another exemplary embodiment, the method involves preventing psoriasis by administering a compound of the invention to an animal, thereby preventing said psoriasis. In another exemplary embodiment, the method involves treating psoriasis by administering a compound of the invention to an animal not otherwise in need of treatment with said compound of the invention, thereby treating said psoriasis. In another exemplary embodiment, the method involves treating psoriasis by administering a therapeutically effective amount of a compound of the invention to an animal not otherwise in need of treatment with said compound of the invention, thereby treating said psoriasis.

In another exemplary embodiment, the method involves preventing plaque psoriasis by administering a compound of the invention to an animal, thereby preventing said plaque psoriasis. In another exemplary embodiment, the method involves treating plaque psoriasis by administering a compound of the invention to an animal not otherwise in need of treatment with said compound of the invention, thereby treating said plaque psoriasis. In another exemplary embodiment, the method involves treating plaque psoriasis by administering a therapeutically effective amount of a compound of the invention to an animal not otherwise in need of treatment with said compound of the invention, thereby treating said plaque psoriasis.

In another exemplary embodiment, the method involves preventing nail psoriasis by administering a compound of the invention to an animal, thereby preventing said nail psoriasis. In another exemplary embodiment, the method involves treating nail psoriasis by administering a compound of the invention to an animal not otherwise in need of treatment with said compound of the invention. In another exemplary embodiment, the method involves treating nail psoriasis by administering a therapeutically effective amount of a compound of the invention to an animal not otherwise in need of treatment with said compound of the invention, thereby treating said nail psoriasis.

In another exemplary embodiment, the method involves treating atopic dermatitis by administering a compound of the invention to an animal, thereby preventing said atopic dermatitis. In another exemplary embodiment, the method involves preventing atopic dermatitis by administering a compound of the invention to an animal not otherwise in need of treatment with said compound of the invention. In another exemplary embodiment, the method involves treating atopic dermatitis by administering a therapeutically effective amount of a compound of the invention to an animal not otherwise in need of treatment with said compound of the invention, thereby treating said atopic dermatitis.

In another aspect, the invention provides a method of treating and/or preventing a disease. The method includes administering to the animal a therapeutically effective amount of the compound of the invention, sufficient to treat and/or prevent the disease. In an exemplary embodiment, the animal being administered the compound is not otherwise in need of treatment with a compound of the invention.

In an exemplary embodiment, the compound is described herein, or a salt, prodrug, hydrate or solvate thereof, or a combination thereof. In an exemplary embodiment, the invention provides a compound described herein, or a salt, hydrate or solvate thereof. In an exemplary embodiment, the invention provides a compound described herein, or a prodrug thereof. In an exemplary embodiment, the invention provides a compound described herein, or a salt thereof. In another exemplary embodiment, the compound of the invention is a compound described herein, or a pharmaceutically acceptable salt thereof. In another exemplary embodiment, the compound is described by a formula listed herein, or a pharmaceutically acceptable salt thereof. In an exemplary embodiment, the compound is part of a pharmaceutical formulation described herein. Such conditions are known to one skilled in the art and specific conditions are set forth in the Examples appended hereto.

In another exemplary embodiment, the animal is a member selected from human, cattle, deer, reindeer, goat, honey bee, pig, sheep, horse, cow, bull, dog, guinea pig, gerbil, rabbit, cat, camel, yak, elephant, ostrich, otter, chicken, duck, goose, guinea fowl, pigeon, swan, and turkey. In another exemplary embodiment, the animal is a human. In another exemplary embodiment, the animal is a mouse. In another exemplary embodiment, the animal is a member selected from goat, pig, sheep, horse, cow, bull, dog, guinea pig, gerbil, rabbit, cat, chicken and turkey. In another exemplary embodiment, the animal is an ungulate. In another exemplary embodiment, the ungulate is selected from the group consisting of horse, zebra, donkey, cattle/bison, rhinoceros, camel, hippopotamus, goat, pig, sheep, giraffe, okapi, moose, elk, deer, tapir, antelope, and gazelle. In another exemplary embodiment, the ungulate is cattle. In another exemplary embodiment, the ungulate is selected from the group consisting of goat, pig, and sheep. In another exemplary embodiment, the animal is a ruminant. In another exemplary embodiment, the ruminant is selected from the group consisting of cattle, goats, sheep, giraffes, bison, yaks, water buffalo, deer, camels, alpacas, llamas, wildebeast, antelope, pronghorn, and nilgai. In another exemplary embodiment, the cattle is a cow. In another exemplary embodiment, the cattle is a bull. In another exemplary embodiment, the cattle is a calf. In another exemplary embodiment, the animal is a snail. In another exemplary embodiment, the animal is an insect. In another exemplary embodiment, the animal is a mosquito. In another exemplary embodiment, the animal is a fly.

In an exemplary embodiment, the disease is treated through oral administration of the compound of the invention. In an exemplary embodiment, the disease is treated through intravenous administration of the compound of the invention. In an exemplary embodiment, the disease is treated through topical administration of the compound of the invention. In an exemplary embodiment, the disease is treated through intraperitoneal administration of the compound of the invention. In an exemplary embodiment, the compound is administered in a topically effective amount. In an exemplary embodiment, the compound is administered in a cosmetically effective amount. In an exemplary embodiment, the pharmaceutical formulation is administered in an orally effective amount.

### VI. Pharmaceutical Formulations

In another aspect, the invention is a pharmaceutical formulation which includes: (a) a pharmaceutically acceptable excipient; and (b) a compound of the invention. In another aspect, the pharmaceutical formulation includes: (a) a pharmaceutically acceptable excipient; and (b) a compound according to a formula described herein. In another aspect, the pharmaceutical formulation includes: (a) a pharmaceutically acceptable excipient; and (b) a compound described herein, or a salt, prodrug, hydrate or solvate thereof. In another aspect, the pharmaceutical formulation includes: (a) a pharmaceutically acceptable excipient; and (b) a compound described herein, or a salt, hydrate or solvate thereof. In another aspect, the pharmaceutical formulation includes: (a) a pharmaceutically acceptable excipient; and (b) a compound described herein, or a salt, hydrate or solvate thereof. In another aspect, the pharmaceutical formulation includes: (a) a pharmaceutically acceptable excipient; and (b) a salt of a compound described herein. In an exemplary embodiment, the salt is a pharmaceutically acceptable salt. In another aspect, the pharmaceutical formulation includes: (a) a pharmaceutically acceptable excipient; and (b) a prodrug of a compound described herein. In another aspect, the pharmaceutical formulation includes: (a) a pharmaceutically acceptable excipient; and (b) a compound described herein. In an exemplary embodiment, the pharmaceutical formulation is a unit dosage form. In an exemplary embodiment, the pharmaceutical formulation is a single unit dosage form.

Information regarding excipients of use in the formulations of the invention can be found in Remington: The Science and Practice of Pharmacy, 21st Ed., Pharmaceutical Press (2011) which is incorporated herein by reference.

Exemplary embodiments are summarized herein below.

In an exemplary embodiment, the invention provides a compound described herein, or a hydrate, solvate, or salt thereof.

In an exemplary embodiment, the invention provides a pharmaceutical formulation comprising: a) the compound described herein, or a pharmaceutically acceptable salt thereof; and b) a pharmaceutically acceptable excipient.

In an exemplary embodiment, the invention provides a method of treating a condition, in an animal, the method comprising administering to the animal a therapeutically effective amount of a compound described herein or a hydrate, solvate, or salt thereof, or a pharmaceutical formulation described herein, thereby treating the condition.

In an exemplary embodiment, according to any of the above paragraphs, the animal is a human.

In an exemplary embodiment, according to any of the above paragraphs, the animal is in need of treatment.

In an exemplary embodiment, according to any of the above paragraphs, the animal is not already in need of treatment by the compound.

In an exemplary embodiment, according to any of the above paragraphs, the invention is a use of a compound described herein or a hydrate, solvate, or salt thereof, or a pharmaceutical formulation described herein in the manufacture of a medicament for the treatment and/or prophylaxis of an anti-inflammatory condition.

The invention is further illustrated by the Examples that follow. The Examples are not intended to define or limit the scope of the invention.

### EXAMPLES

The following Examples illustrate the synthesis of representative compounds used in the invention and the following Reference Examples illustrate the synthesis of intermediates in their preparation. These examples are not intended, nor are they to be construed, as limiting the scope of the invention. It will be clear that the invention may be practiced otherwise than as particularly described herein. Numerous modifications and variations of the invention are possible in view of the teachings herein and, therefore, are within the scope of the invention.

All temperatures are given in degrees Centigrade. Room temperature means 20 to 25° C. Reagents were purchased from commercial sources or prepared following standard literature procedures. Unless otherwise noted, reactions were carried out under a positive pressure of nitrogen. Reaction vessels were sealed with either rubber septa or Teflon screw caps. Nitrogen was introduced through Tygon tubing, fitted with a large bore syringe needle. Concentration under vacuum refers to the removal of solvent on a Büchi Rotary Evaporator.

Analytical HPLC was performed using a Supelco discovery Cis 15 cm×4.6 mm/5 μm column coupled with an Agilent 1050 series VWD UV detector at 210 nm. Conditions: Solvent A: H₂O/1% acetonitrile/0.1% HCO₂H; Solvent B: methanol.

Proton magnetic resonance (¹H NMR) spectra were recorded on a Varian INOVA NMR spectrometer [400 MHz (¹H) or 500 MHz (¹H)] or Varian 400-MR [400 MHz (1H)]. All spectra were determined in the solvents indicated. Although chemical shifts are reported in ppm downfield of tetramethylsilane, they are referenced to the residual proton peak of the respective solvent peak for ¹H NMR. Interproton coupling constants are reported in Hertz (Hz).

LCMS spectra were obtained using a ThermoFinnigan AQA MS ESI instrument utilizing a Phenomenex Aqua 5 micron Cis 125 Å 50×4.60 mm column. The spray setting for the MS probe was at 350 μL/min with a cone voltage at 25 mV and a probe temperature at 450° C. The spectra were recorded using ELS and UV (254 nm) detection. Alternatively, LCMS spectra were obtained using an Agilent 1200SL HPLC equipped with a 6130 mass spectrometer or Agilent 1200 series with a 6140 mass spectrometer operating with electrospray ionization.

Silica gel chromatography was carried out on either a Teledyne ISCO CombiFlash Companion or Companion Rf Flash Chromatography System with a variable flow rate from 5-100 mL/min. The columns used were Teledyne ISCO RediSep Disposable Flash Columns (4, 12, 40, 80, or 120 g prepacked silica gel), which were run with a maximum capacity of 1 g crude sample per 10 g silica gel. Samples were preloaded on Celite in Analogix Sample Loading Cartridges with frits (1/in, 1/out). The eluent was 0-100% EtOAc in heptane or 0-10% MeOH in CH₂Cl₂ as a linear gradient over the length of the run (14-20 minutes). Peaks were detected by variable wavelength UV absorption (200-360 nm). The resulting fractions were analyzed, combined as appropriate, and evaporated under reduced pressure to provide purified material.

### Example 1

### A) 4-(1-hydroxy-1,3-dihydrobenzo[c][1,2]oxaborole-5-carbonyl)benzonitrile

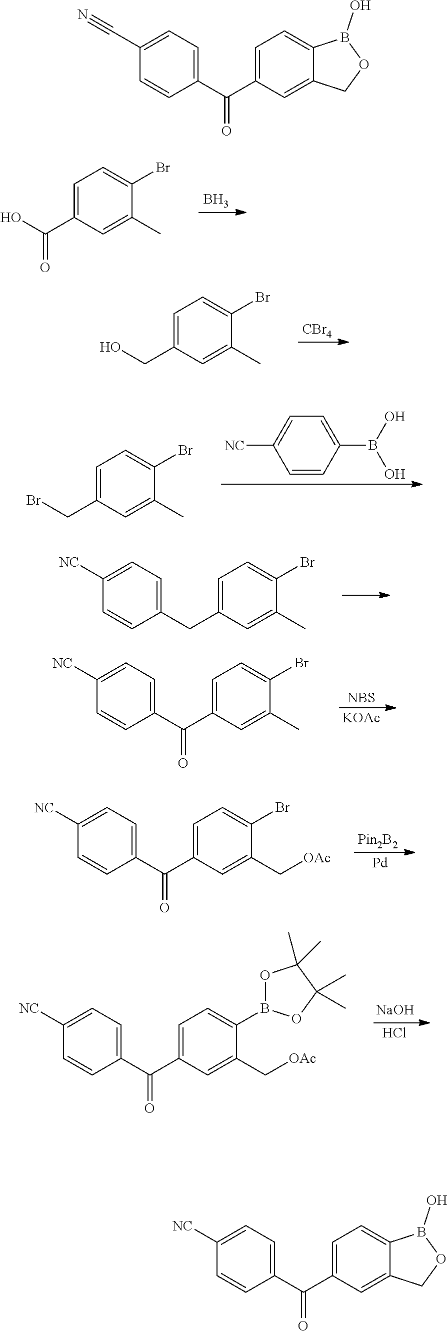

### Preparation of (4-bromo-3-methylphenyl)methanol

To a solution of 4-bromo-3-methylbenzoic acid (30 g, 0.14 mol) in THF 300 mL) was added borane (10 Min THF, 70 mL, 0.7 mol) dropwise at 0° C. and the mixture was stirred at room temperature overnight. The reaction was quenched with methanol and water (10 mL). After removal of the solvent by rotary evaporation, the residue was extracted with dichloromethane. The organic layer was washed with brine, dried over Na₂SO₄ and concentrated to give (4-bromo-3-methylphenyl)methanol (27.5 g, 98%). ¹H NMR (400 MHz, CDCl₃) δ 7.52-7.50 (d, 1H), 7.25 (s, 1H), 7.06-7.04 (d, 1H), 4.63 (s, 2H), 2.41 (s, 3H).

### Preparation of 1-bromo-4-(bromomethyl)-2-methylbenzene

To a solution of (4-bromo-3-methylphenyl)methanol (27.5 g, 136.8 mmol) in dichloromethane (250 mL) was added PPh₃ (39.4 g, 150.5 mmol) and CBr₄ (49.9 g, 150.5 mmol) and the mixture was stirred for 2 hrs at room temperature. Water was added. The organic layer was separated, dried over anhydrous sodium sulfate and concentrated. The crude product was purified by chromatography to give 1-bromo-4-(bromomethyl)-2-methylbenzene (34 g, 94%). ¹H NMR (400 MHz, CDCl₃) δ 7.94-7.92 (d, 1H), 7.70 (s, 1H), 7.53-7.51 (d, 1H), 4.86 (s, 2H), 2.83 (s, 3H).

### Preparation of 4-(4-bromo-3-methylbenzyl)benzonitrile

A mixture of 1-bromo-4-(bromomethyl)-2-methylbenzene (12.0 g, 45.4 mmol), 4-cyanophenylboronic acid (6.00 g, 40.9 mmol), AcOK (8.9 g, 90.8 mmol) and Pd(PPh₃)₂C₂ (1.6 g, 2.27 mmol) in dry dioxane (120 mL) was heated to 80° C. overnight. Then the mixture was poured into cold water and extracted with EtOAc. The combined organic layer was washed with brine, dried over Na₂SO₄ and concentrated in vacuo. The residue was purified by column chromatography to give 4-(4-bromo-3-methylbenzyl)benzonitrile (8.6 g, 65%) as a solid. ¹H NMR (400 MHz, CDCl₃) δ 7.59-7.57 (d, 1H), 7.47-7.45 (d, 1H), 7.28-7.26 (m, 3H), 7.03 (s, 1H), 6.86-6.84 (d, 1H), 3.95 (s, 2H), 2.36 (s, 3H).

### Preparation of 4-(4-bromo-3-methylbenzoyl)benzonitrile

To a mixture of 4-(4-bromo-3-methylbenzyl)benzonitrile (10 g, 34.9 mmol) in acetic anhydride (60 mL) was added chromium trioxide (28 g, 0.28 mol) in portions and the mixture was stirred 30 min. Water was added and the mixture was extracted with dichloromethane. The combined organic layer was washed with brine, dried over Na₂SO₄ and concentrated in vacuo. The residue was purified by column chromatography to give 4-(4-bromo-3-methylbenzoyl)benzonitrile (3.6 g, 34%) as a solid. ¹H NMR (400 MHz, CDCl₃) δ 7.87-7.79 (m, 4H), 7.69-7.66 (m, 2H), 7.44-7.42 (d, 1H), 2.48 (s, 3H).

### Preparation of 2-bromo-5-(4-cyanobenzoyl)benzyl acetate

A mixture of 4-(4-bromo-3-methylbenzoyl)benzonitrile (3.6 g, 12 mmol), NBS (2.13 g, 12 mmol) and AIBN (197 mg, 1.2 mmol) in CCl₄ (80 mL) was heated to reflux overnight. Then the mixture was concentrated in vacuo, the residue was diluted with DCM and washed with water and brine, dried over Na₂SO₄ and concentrated. The residue was dissolved in DMF (30 mL) was added AcOK (3.5 g, 36 mmol) in portions and stirred for 1 hr at 25° C. Then the mixture was poured into cold water and extracted with EtOAc. The combined organic layer was washed with brine, dried over Na₂SO₄ and evaporated to dryness. The residue was purified by column chromatography to 2-bromo-5-(4-cyanobenzoyl)benzyl acetate (2.3 g, 53%) as a solid. ¹H NMR (400 MHz, CDCl₃) δ 7.88-7.81 (m, 5H), 7.75-7.73 (d, 1H), 7.59-7.57 (d, 1H), 5.24 (s, 2H), 2.15 (s, 3H).

### Preparation of 5-(4-cyanobenzoyl)-2-(4,4,5,5-tetramethyl-1,3,2-dioxaborolan-2-yl)benzyl acetate

A mixture of 2-bromo-5-(4-cyanobenzoyl)benzyl acetate (2.8 g, 7.8 mmol), bis(pinacol)diborane (2.4 g, 9.4 mmol), AcOK (2.3 g, 23.4 mmol) and Pd(dppf)Cl₂ (285 mg, 0.39 mmol) in dry dioxane (250 mL) was heated to 80° C. overnight. The solvent was removed and the residue was purified by column chromatography to give 5-(4-cyanobenzoyl)-2-(4,4,5,5-tetramethyl-1,3,2-dioxaborolan-2-yl)benzyl acetate (2.9 g, 91%) as a solid. ¹H NMR (400 MHz, CDCl₃) δ 7.98-7.96 (d, 1H), 7.88-7.86 (d, 2H), 7.81-7.78 (m, 3H), 7.67-7.65 (d, 1H), 5.42 (s, 2H), 2.09 (s, 3H), 1.37 (s, 12H).

### Preparation of A

To a solution of 5-(4-cyanobenzoyl)-2-(4,4,5,5-tetramethyl-1,3,2-dioxaborolan-2-yl)benzyl acetate (2.7 g, 6.7 mmol) in THF (30 mL) was added dropwise NaOH (532 mg, 13.3 mmol) in water (30 mL) with ice bath. After addition, the mixture was stirred for 4 hrs at room temperature, then diluted with water, adjusted to pH=3 with 2.0 M HCl and extracted with EtOAc. The combined organic layer was washed with brine, dried over Na₂SO₄ and concentrated in vacuo. The residue was purified by Preparative-HPLC to give A, 4-(1-hydroxy-1,3-dihydrobenzo[c][1,2]oxaborole-5-carbonyl)benzonitrile (1.2 g, 68.6%) as a white solid. ¹H NMR (400 MHz, DMSO-d₆) δ 9.47 (s, 1H), 8.06-8.04 (d, 2H), 7.92-7.88 (m, 3H), 7.78 (s, 1H), 7.73-7.71 (d, 1H), 5.07 (s, 2H).

### B) 4-(hydroxy(1-hydroxy-1,3-dihydrobenzo[c][1,2]oxaborol-5-yl)methyl)benzonitrile

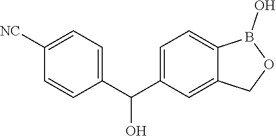 [SMILES: N#Cc1ccc(C(O)c2ccc3c(c2)COB3O)cc1]

To a solution of 4-(1-hydroxy-1,3-dihydrobenzo[c][1,2]oxaborole-5-carbonyl)benzonitrile (500 mg, 1.9 mmol) in MeOH (20 ml) was added NaBH₄ (108 mg, 2.8 mmol) at 0° C. and the mixture was stirred for 1 h. Then the mixture was adjusted to pH=2-3 with 2 M HCl. The solvent was removed and the residue was added water and EtOAc. The organic layer was washed with brine, dried over Na₂SO₄ and concentrated to give the crude B, 4-(hydroxy(1-hydroxy-1,3-dihydrobenzo[c][1,2]oxaborol-5-yl)methyl)benzonitrile, which was purified by Preparative-HPLC (400 mg, yield 71%) as white solid. ¹H NMR (400 MHz, DMSO-d₆) δ 9.13 (s, 1H), 7.78-7.76 (d, 2H), 7.67-7.65 (d, 1H), 7.61-7.59 (d, 2H), 7.43 (s, 1H), 7.37-7.35 (d, 1H), 6.23 (s, 1H), 5.85 (s, 1H), 4.95 (s, 2H).

### C) 4-((1-hydroxy-1,3-dihydrobenzo[c][1,2]oxaborol-5-yl)methyl)benzonitrile

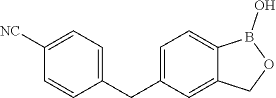 [SMILES: N#Cc1ccc(Cc2ccc3c(c2)COB3O)cc1]

To a mixture of 4-(hydroxy(1-hydroxy-1,3-dihydrobenzo[c][1,2]oxaborol-5-yl)methyl)benzonitrile (0.4 g, 1.5 mmol) in TFA/DCM (10/10 mL) was added Et₃SiH (870 mg, 7.5 mmol) dropwise. The solution was stirred for 48 hrs. The pH was adjusted to around 6 by progressively adding solid NaOH. The solvent was removed. The residue was purified by Preparative-HPLC to give C, 4-((1-hydroxy-1,3-dihydrobenzo[c][1,2]oxaborol-5-yl)methyl)benzonitrile (111 mg, yield: 29.5%). ¹H NMR (400 MHz, DMSO-d₆) δ 9.10 (s, 1H), 7.77-7.75 (d, 2H), 7.67-7.65 (d, 1H), 7.47-7.45 (d, 2H), 7.27-7.23 (m, 2H), 4.94 (s, 2H), 4.10 (s, 2H).

### D) 4-(1-hydroxy-1,3-dihydrobenzo[c][1,2]oxaborol-5-ylsulfinyl)benzonitrile

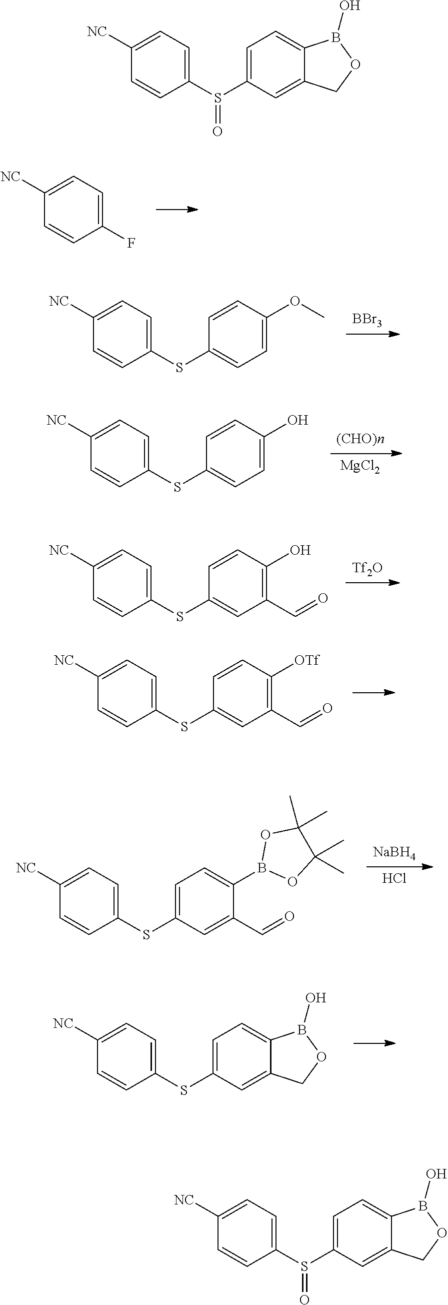

### Preparation of 4-(4-methoxyphenylthio)benzonitrile

To a solution of 4-fluorobenzonitrile (1.21 g, 10.0 mmol) and 4-methoxythiophenol (1.47 g, 10.5 mmol) in DMF (50 mL) was added Cs₂CO₃ (6.50 g, 20 mmol) at room temperature under N₂. The mixture was stirred at 80° C. for 5 hours. The mixture was diluted by water, extracted by EtOAc. The separated organic layer was concentrated in vacuo and the residue was purified by column chromatography (Petroleum ether/EtOAc:5/1) to afford 4-(4-methoxyphenylthio)benzonitrile (2.1 g, yield: 87%) as light yellow solid. ¹H NMR (400 MHz, CDCl₃) δ 7.49-7.44 (m, 4H), 7.08 (d, J=8.4 Hz, 2H), 6.98 (d, J=8.4 Hz, 2H), 3.87 (s, 3H).

### Preparation of 4-(4-hydroxyphenylthio)benzonitrile

Boron tribromide (50 mL of a 1.0 M solution in CH₂Cl₂, 50 mmol) was added to a stirred solution of 4-(4-methoxyphenylthio)benzonitrile (2.4 g, 10 mmol) in dry CH₂Cl₂ (100 mL) under nitrogen at 0° C., and then the mixture was stirred at room temperature for 2.5 h. The resulting solution was added to a mixture of ice and aqueous sodium bicarbonate and extracted with EtOAc. The organic layer was washed with water and evaporated to dryness, and then the residue was purified by column chromatography (Petroleum ether/EtOAc:3/1) to afford 4-(4-hydroxyphenylthio)benzonitrile (1.9 g, 84%) as white solid. ¹H NMR (400 MHz, CDCl₃) δ 9.42 (s, H), 7.50-7.43 (m, 4H), 7.08 (d, J=4.8 Hz, 2H), 6.98 (d, J=4.8 Hz, 2H).

### Preparation of 4-(3-formyl-4-hydroxyphenylthio)benzonitrile

To a solution of 4-(4-hydroxyphenylthio)benzonitrile (2.3 g, 10 mmol) in CH₃CN (50 mL) was added paraformaldehyde (2.1 g, 70 mmol), MgCl₂ (1.96 g, 20 mmol) and triethylamine (4.04 g, 40 mmol) at room temperature under N₂. The mixture was stirred at reflux overnight. Then added NH₄Cl (aq) and extracted with EtOAc, concentrated to give the crude product. The residue was used for next step.

### Preparation of Compound 4-(4-cyanophenylthio)-2-formylphenyl trifluoromethanesulfonate

To a solution of the crude product obtained above (2.6 g, 10 mmol) and triethylamine (2.53 g, 25 mmol) in dry CH₂Cl₂ (100 mL) was added Tf₂O (7.05 g, 25 mmol) at −30° C. The reaction mixture was stirred at room temperature for 30 min. Then added NH₄Cl (aq) and extracted with EtOAc, concentrated to give the crude product. The residue was purified by column chromatography (Petroleum ether/EtOAc:10/1) to afford 4-(4-cyanophenylthio)-2-formylphenyl trifluoromethanesulfonate (1.3 g, 34%) as white solid. ¹H NMR (400 MHz, CDCl₃) δ 10.25 (s, H), 7.99 (s, H), 7.69 (d, J=7.8 Hz, 1H), 7.62 (d, J=8.0 Hz, 2H), 7.43 (d, J=8.4 Hz, 1H), 7.38 (d, J=8.4 Hz, 2H).

### Preparation of 4-(3-formyl-4-(4,4,5,5-tetramethyl-1,3,2-dioxaborolan-2-yl)phenylthio)benzonitrile

To a solution of 4-(4-cyanophenylthio)-2-formylphenyl trifluoromethanesulfonate (1.30 g, 3.36 mmol), bis(pinacolato)diboron (1.02 g, 4.02 mmol) and KOAc (0.99 g, 10.1 mmol) in 1,4-dioxane (50 mL) was added Pd(dppf)Cl₂ (0.25 g, 0.36 mmol) at room temperature under N₂. The mixture was heated at 80° C. overnight. The mixture was evaporated to dryness, flash chromatography (Petroleum ether/EtOAc: 10/1) to give 4-(3-formyl-4-(4,4,5,5-tetramethyl-1,3,2-dioxaborolan-2-yl)phenylthio)benzonitrile (0.90 g, 73%).

### Preparation of 4-(1-hydroxy-1,3-dihydrobenzo[c][1,2]oxaborol-5-ylthio)benzonitrile

A solution of compound 6 (0.9 g, 2.46 mmol) in MeOH (50 mL) was added NaBH₄ (0.37 g, 9.86 mmol) at 0° C. The mixture was stirred at room temperature over 1 h. The mixture was acidified with HCl to pH=2 and concentrated to give crude product which was purified by pre-HPLC to afford 4-(1-hydroxy-1,3-dihydrobenzo[c][1,2]oxaborol-5-ylthio)benzonitrile (0.22 g, 34%) as off-white solid. ¹H NMR: TH05922-123-2B (400 MHz DMSO) δ 9.36 (s, 1H), 7.81 (d, J=8.0 Hz, 1H), 7.76 (d, J=8.4 Hz, 2H), 7.57 (s, 1H), 7.46 (d, J=7.6 Hz, 1H), 7.32 (d, J=8.4 Hz, 2H), 5.00 (s, 2H).

### Preparation of D

To a solution of the crude 4-(1-hydroxy-1,3-dihydrobenzo[c][1,2]oxaborol-5-ylthio)benzonitrile (0.9 g, 3.37 mmol) in H₂O: MeOH=10% (75 mL, v/v) was added NaIO₄ (2.16 g, 10 mmol). The reaction mixture was stirred at room temperature overnight, then evaporated and extracted with EtOAc. The organic layer was washed with water and brine, and dried over anhydrous Na₂SO₄. After rotary evaporation the residue was purified by preparative-HPLC to afford D, 4-(1-hydroxy-1,3-dihydrobenzo[c][1,2]oxaborol-5-ylsulfinyl)benzonitrile (0.12 g, 13%) as off-white solid. ¹H NMR (400 MHz, DMSO-d₆) δ 9.40 (s, 1H), 8.02 (d, J=8.4 Hz, 2H), 7.95 (d, J=8.0 Hz, 2H), 7.88-7.86 (m, 2H), 7.74 (d, J=7.2 Hz, 1H), 5.03 (s, 2H).

### E) 4-(1-hydroxy-1,3-dihydrobenzo[c][1,2]oxaborol-5-ylsulfinyl)benzonitrile

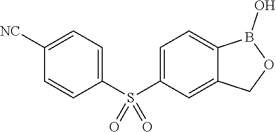 [SMILES: N#Cc1ccc(S(=O)(=O)c2ccc3c(c2)COB3O)cc1]

To a solution of the crude 4-(1-hydroxy-1,3-dihydrobenzo[c][1,2]oxaborol-5-ylthio)benzonitrile (0.6 g, 2.24 mmol) in H₂O:MeOH=10% (50 mL, v/v) was added NaIO₄ (2.4 g, 11 mmol). The reaction mixture was stirred at 60° C. overnight, then evaporated and extracted with EtOAc. The organic layer was washed with water and brine, and dried over anhydrous Na₂SO₄. After rotary evaporation the residue was purified by preparative-HPLC to afford E, 4-(1-hydroxy-1,3-dihydrobenzo[c][1,2] oxaborol-5-ylsulfinyl)benzonitrile (0.09 g, 13%) as off-white solid. ¹H NMR (400 MHz, DMSO-d₆) δ 9.53 (s, 1H), 8.17-8.09 (m, 5H), 7.97 (s, 2H), 5.07 (s, 2H).

### Example 2

### Measurement of Phosphodiesterase 4 (PDE4) Inhibition

Human PDE4 enzymatic activity was assayed using the zinc and barium precipitation method of Saldou, N. et al (1998: Cell Signal, vol 10, No. 6, pp 427-440). The assay was performed in 96-well v-bottom plates (BrandPlates, Wertheim, Germany). PDE4B₂ truncated is the catalytic domain including the UCR2 and a portion of the UCR1 domain of PDE4B2 (23.4 mg/mL) from Proteros Biostructures. Final concentration of enzyme in the assay was 0.3 pM. Stocks of the tested compounds were prepared in 100% DMSO at a concentration of 40 mM. The 100-μL reaction volume consisted of 45 mM Tris-HCl, pH 7.5, 12.5 mM MgSO4, 0.3 μM AMP (Sigma, St Louis, Mo.) supplemented with 0.2 μM ³H-cAMP (Perkin Elmer, Waltham, Mass.). Compounds were pre-incubated with enzyme at room temperature for 20 min prior to initiating the reaction by adding substrate (cAMP supplemented with ³H-cAMP). After incubation at 30° C. for 6 minutes, the reaction was stopped by the addition of 50 μL fresh made 21.5 mM ZnSO₄.7H₂O (Alfa Aesar, Ward Hill, Mass.) containing 5 mM AMP as a co-precipitant. Total AMP was precipitated by the addition of 50 μL 17.5 mM Ba(OH)₂, prepared fresh daily in water. The precipitate was transferred and collected by filtration onto a 96-well GF/B plate (Millipore, Ireland). The plates were washed five times with cold wash solution (1 mM NaOH, 100 mM NaCl) and dried at 65° C. The bottoms of plates were sealed with clear tapes (Millipore Co-operation, Billerica, Mass.) and 40 μL of scintillation cocktail (Perkin Elmer Waltham, Mass.). The plates were top sealed and radioactivity was measured with an Elmer Packard TopCount scintillation counter.

Data for compounds of the invention are provided in the table below:

| | PDE4 |
|---|---|
| | IC50 (μM) |
| A | 0.504 |
| B | 1.732 |
| C | 1.774 |
| D | 0.062 |
| E | 2.062 |

It is understood that the examples and embodiments described herein are for illustrative purposes only and that various modifications or changes in light thereof will be suggested to persons skilled in the art and are to be included within the spirit and purview of this application and scope of the appended claims. It is to be understood that the present invention covers all combinations of aspects and/or embodiments, as well as suitable, convenient and preferred groups described herein. All publications, patents, and patent applications cited herein are hereby incorporated by reference in their entirety for all purposes.

The invention is further illustrated by the Examples that follow. The Examples are not intended to define or limit the scope of the invention.

What is claimed is:

1. A compound described herein, or a hydrate, solvate, or salt thereof.

2. A pharmaceutical composition comprising:
a) the compound of claim 1, or a hydrate, solvate, or pharmaceutically acceptable salt thereof; and
b) a pharmaceutically acceptable excipient.

3. A method of treating a condition in an animal, comprising:
a) administering a compound or pharmaceutical formulation of a preceding claim to the animal,
thereby treating the condition in the animal.

4. The method of claim 3, wherein the animal is a human.
